(19) United States
(12) Patent Application Publication
(10) Pub. No.: US 2009/0230058 A1
(43) Pub. Date: Sep. 17, 2009

(54) CONTROL OF BUBBLE FORMATION IN EXTRACORPOREAL CIRCULATION

(76) Inventor: Fredrik Boris-Moeller, Lund (SE)

Correspondence Address: MICHAEL J. STRIKER, 103 EAST NECK ROAD, HUNTINGTON, NY, 11743, US

(21) Appl. No.: 11/909,138

(22) PCT Filed: Mar. 16, 2006
(86) PCT No.: PCT/SE06/50032

§ 371 Date: Sep. 19, 2007

(30) Foreign Application Priority Data
Mar. 24, 2005 (SE) 0500662-2

(51) Int. Cl. A61M 1/34; B01D 61/32

(52) U.S. Cl. 210/637; 422/47; 210/647

(57) ABSTRACT

The invention relates to control of bubble formation in a fluid during extracorporeal circulation. A fluid supply means (111a) is configured to supply fluid to the extracorporeal circuit (111a, 111b, 113, 114, 115, 116), a flow control means (113) is connectable to the extracorporeal circuit and configured to control the flow of the fluid in the circuit; a gas exchange means (114) is connectable to the circuit and configured to gas exchange of the circulated fluid; an antibubble control unit (125) is connectable to an outlet (123) of the gas exchange means and configured to control the total gas pressure over a gas-exchange membrane (118) of the gas exchange means, whereby the amount of gas in the fluid leaving the gas exchange means can be controlled; and fluid return means (115, 116) is connected to the gas exchange means and configured to reintroduce the fluid into the patient.

### TECHNICAL FIELD

The present invention relates to control of bubble formation in a body fluid during extracorporeal circulation. More precisely, the present invention relates to minimization of the bubble formation.

### BACKGROUND OF THE INVENTION

Primarily during heart operations there is a transient need to replace the function of the heart and lungs by artificial means. Also in more chronic disease states as e.g. during severe pulmonary, cardiac, or renal failure, maintenance of life can be upheld by different artificial means until an organ for transplantation becomes available. In many clinical situations there is a need for an extracorporeal circuit wherein the artificial organ is incorporated.

The contact of blood on surfaces made out of foreign material inevitably initiates blood coagulation and the formation of clots. This is controlled by the use of anticoagulant drugs. Also gas bubbles are easily formed in blood, which is propelled into the circulation of a living being during extracorporeal circulation. This phenomenon is due to cavitation, temperature gradients, and differences in the amount of gases dissolved between own and incoming blood. In the case of heart surgery the extracorporeal circuit contains a gas-exchange device i.e. an oxygenator, which is used not only for oxygenation but also for the disposal of carbon dioxide. The close contact between blood and gas in the oxygenator poses even higher risks for inadvertent entry of gas bubbles into the circulating blood.

At present, the avoidance of bubble formation during heart surgery include the change of the clinical use of bubble-oxygenators into the membrane-type, the avoidance of high temperature gradients, and a controlled use of suction in the operating field. All heart-lung machines contain an air bubble sensor that warns the perfusionist, i.e. the person maneuvering the heart-lung machine, of the appearance of small bubbles and immediately stops the main pump when larger bubbles appear. Typically, the bubble sensor can discern bubbles with a diameter of approximately 0.3 mm, but stops the main pump first when a bubble with a diameter of 3-5 mm is recognized.

There are numerous technical solutions in the prior art to separate already formed bubbles from circulation. In the patent document U.S. Pat. No. 5,362,406 a method is disclosed using a porous sponge material for inducing small bubbles to coalesce and the formed larger bubbles are then subsequently vented from the extracorporeal circuit. Similar in construction are the filtering devices disclosed in the patent document U.S. Pat. No. 6,328,789 B1. The patent document U.S. Pat. No. 6,478,962 discloses a method for bubble separation by strong radial acceleration forces thus concentrating bubbles to the center of the accelerated blood flow.

However, there is no device available intended to diminish the generation of gas bubbles i.e. the formation of gas bubbles during e.g. heart surgery. In a blood bubble, in the liquid-gas interface, there is an approximately 40-100 Å (i.e. 4-10 nanometer) deep layer of lipoproteins that denaturate due to direct contact with the foreign material, e.g. gas. In turn, the Hageman factor is activated which initiates coagulation and the concomitant adverse consumption of factors promoting coagulation, which in the post-pump period are desperately needed to prevent bleeding from the surgical wound. It seems therefore more beneficial and logical to inhibit the bubble formation in the blood during extracorporeal circulation rather than to allow bubble formation and subsequent compulsory removal of bubbles.

To inhibit bubble formation in a liquid, the method of lowering the partial pressures of dissolved gases in the liquid has been employed for industrial design. The US patent document 2003/0205831 A1 discloses a method for the use in vehicle glass repair. A vacuum pump coupled to the repair space degasses both the damaged area and the repair material. This method is not intended for circulating fluids and cannot be used in extra-corporeal circulation during e.g. heart surgery.

The method disclosed in U.S. Pat. Nos. 5,772,736; 5,645,625; 5,425,803, and EP 0 598 424 A3 is used on moving liquids and its purpose is to eliminate large overpressure of a dissolved propellant gas such as helium. An application of the degassing module as described in these patents in extracorporeal circulation would be of no avail, since the pressure of dissolved gas in the liquid is reduced only to ambient atmospheric pressure—a situation already present in the oxygenator of any heart-lung machine setup.

The patent document WO02/100510 A1 discloses a method to degas preferably water by applying vacuum over a gas-permeable membrane letting the liquid flow on the other side. The problem solved with this method is how to generate vacuum without great loss of water, since water is used in the high stream ejector type production of vacuum, based on Bernoulli's principle. A problem with this setup is that the returned water used for vacuum generation becomes supersaturated with the removed gas and it is supposed that time will provide equilibrium of dissolved gas in the returned water with the ambient air and thus diminish excess gas before return to the reservoir. To implement this method on blood would be hazardous, most probably due to bubble formation in the returned, initially gas-supersaturated blood but also due to the blood injury that would ensue during the vigorous pumping of blood for generation of vacuum.

The U.S. Pat. No. 6,596,058 discloses a method for degassing the mobile phase in high performance liquid chromatography. The method utilizes application of reduced pressure or vacuum to the solvent over a liquid-impermeable and gas-permeable membrane. This document is focused on the manufacturing, without supporting structures, of the gas-permeable membrane dividing the fluid and vacuum portions of the degassing chamber.

### PURPOSE OF THE INVENTION

The purpose of the present invention is to control, and especially to minimize, bubble formation and bubble size in a body fluid during extracorporeal circulation of a living being.

An aspect is to control dissolved gas in the fluid in an extracorporeal circulation circuit.

### BRIEF DESCRIPTION OF THE INVENTION

The purpose of the invention is fulfilled by a system, a method and apparatus according to the independent claims. Preferred embodiments of the invention are set out in the dependent claims.

The invention fulfills the purpose by decreasing the total gas pressure of the fresh gas distributed to a gas exchanging compartment of a gas exchange means comprised in an extracorporeal circuit. This can for example be accomplished by a combination of a) the complete airtight closure of the gas compartment of the gas exchange means except for the gas inlet(s) and outlet(s); b) connecting a fresh gas tubing to the gas inlet(s) of the gas exchange means through gastight and incollapsable tubes; c) safeguarding against an inadvertent overpressurization of the gastight constructed gas exchange means by e.g. safety valve(s); d) providing an alerting alarm device for warning the user against too high a pressure difference over the gas-exchange membrane; e) attaching through gastight and incollapsable tubes a suction apparatus to the gas outlet(s) for the control of the total gas pressure over the gas-exchange membrane in the gas exchange means, thus controlling the dissolved amount of gas of the blood leaving the gas exchange means; f) and/or connecting the gas exhaust tubing of the suction device mentioned under e) to a gas outlet so that the appropriate disposal of volatile anesthetic gases can be performed.

The subatmospheric pressure in the above described airtight gas exchange means will not only extract dissolved gas of the blood but also induce formed bubbles entering into the gas exchange means to increase in volume proportionally. Since there is formation of a denatured layer of lipoproteins in the gas-liquid interface of blood, the transient volume increase of a bubble passing through a gas exchange means with subatmospheric pressure may enlarge the total bubble surface area and thus the total amount of irreversibly denatured lipoprotein. This can be counteracted by the application of a proportionally increased hydrostatic blood pressure in the blood compartment of the gas exchange means.

Furthermore, by increasing the hydrostatic pressure of a liquid containing bubbles it is possible to force gas molecules from the gas bubble into a soluble state in the liquid. Hydrostatic pressure high enough may even annihilate the bubble completely. In one embodiment of the invention, a supplementary device for the temporary increase of hydrostatic pressure is incorporated for this purpose in the tubing of the extracorporeal circuit, preferably between the flow control means and the gas exchange means.

### DESCRIPTION OF THE DRAWINGS

The present invention will be described in further detail below, with reference to the accompanying drawing, in which:

FIG. 1 schematically illustrates a first exemplifying embodiment of the invention;

FIG. 2 schematically illustrates a second exemplifying embodiment of the invention; and

FIG. 3 schematically illustrates a third exemplifying embodiment of the invention.

### DETAILED DESCRIPTION OF THE INVENTION

The present invention relates to a system, an apparatus, and a method for controlling the bubble formation in an extracorporeal circulatory procedure. It is intended for heart surgery, but can also be employed in a multitude of clinical applications, e.g. dialysis, in which it is desirable to extracorporeally circulate a body fluid. Thus the oxygenator can be modified according to different clinical prerequisites and the flow can also be generated by the arterial-venous pressure difference rather than a pump.

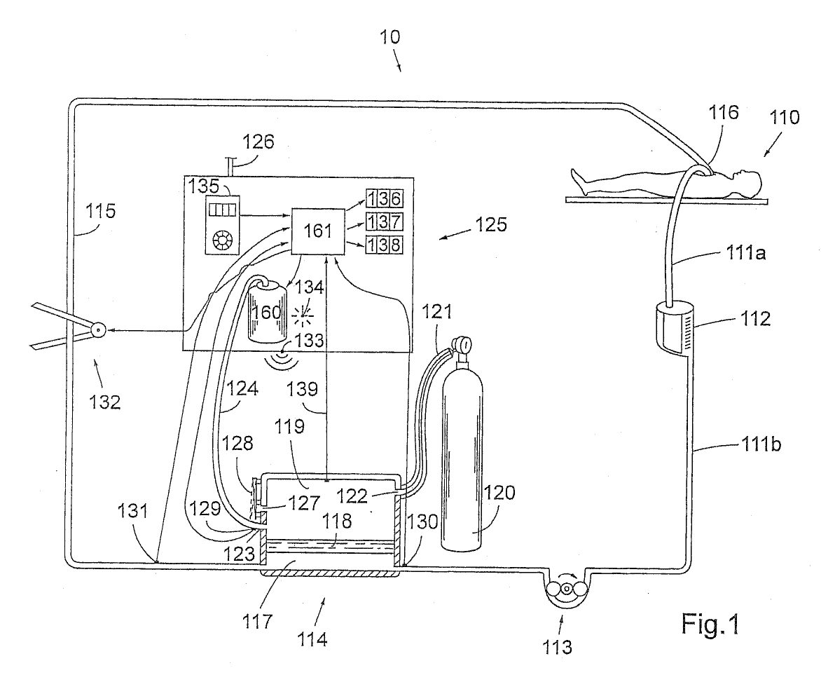

Exemplifying embodiments of the present invention will be described in more detail with reference to FIGS. 1-3, in which the same reference numerals are used for the same or similar components or features.

FIG. 1 schematically depicts a first embodiment of a system according to the present invention, which system 10 can be used in e.g. open heart surgery. The figure shows how the application of vacuum to a gas exchange means, such as an oxygenator, can be employed and also how an increased hydrostatic blood pressure during blood passage through the oxygenator can be generated according to the invention and setup of the device during extracorporeal perfusion.

An embodiment of the inventive system 10, according to FIG. 1, comprises tubings 111a by means of which venous blood can be diverted from a patient 110 to an extracorporeal venous reservoir 112. In this description, the tubing 111a will also be referred to as a venous line 111a. However it should be understood that the tubing also can be an arterial line in applications where arterial blood is to be withdrawn from the patient. The venous reservoir 112 is configured to collect, by gravity force or by an applied subatmospheric pressure, the venous blood from the patient. Also blood sucked from an operating field can be reused by repumping it into the reservoir 112. Large gas bubbles in blood that enter the venous reservoir 112 will rise by gravitation to the surface and are thus disposed of, since the reservoir is open to ambient air or an applied subatmospheric pressure.

The system can further comprise a flow control means 113 arranged to generate propellant energy to the blood withdrawn from the patient 110, whereby the withdrawn blood is circulated in an extracorporeal circuit. The flow control means 113 can for example be a pumping means realized as a main pump 113 of a heart-lung machine (not shown). As illustrated in FIG. 1, the flow control means 113 is arranged at tubings 111b between the reservoir 112 and a gas exchange means 114. In the present description text and in order to exemplify the invention, reference will be made to an oxygenator 114. However, it should be understood that the gas exchange means can be realized as another kind of device capable of diminishing and/or exchanging gas comprised in a fluid.

The oxygenator 114 is connected or connectable to the extracorporeal circuit and as shown in the figures, the oxygenator 114 is in the shown embodiment arranged downstream the reservoir 112 and the flow control means 113. The oxygenator 114 is configured to provide gas exchange of the withdrawn blood circulated in the extracorporeal circuit. The extracorporeal circuit comprises further tubings 115 by means of which oxygenated blood flows from the oxygenator 114 back to the patient 110. In the embodiment shown in FIG. 1, wherein venous blood is withdrawn, the blood flows back to the patient 110 via an arterial line 115 and an arterial cannula 116 inserted into an artery of the patient 110, however it should be understood that the tubing also can be a venous line in applications where arterial or venous blood is to be withdrawn from the patient.

The oxygenator 114 comprises a first compartment 117 also called the blood compartment 117 when blood is the fluid flowing in the circuit. The oxygenator 114 comprises further a gas-fluid separating membrane or a gas-blood separating membrane 118 through which the gas exchange and exhaust occurs, and a second compartment 119 also called the gas compartment 119.

The system comprises also a gas source 120 by means of which fresh gas is supplied to the oxygenator 114 via a gas supply tube 121 and a gas inlet 122 of the second compartment 119 of the oxygenator 114. The fresh gas can for example be a mixture of oxygen, nitrogen, and a volatile anesthetic agent and is supplied after pressure reduction via a gas flow-meter from the gas source 120. The membrane 118 is configured permeable to the supplied fresh gas, whereby gas exchange between fresh gas and venous blood occurs since the partial pressures of gases in the gas compartment 119 and the partial pressures of gases dissolved in blood passing through the blood compartment 117 tend to equalize. After having passed the gas compartment 119 of the oxygenator 114, the gas flow is directed to a gas outlet 123 of the oxygenator 114.

According to the present invention, the gas flows via a gas outlet tubing 124 connected to the gas outlet 123 to an antibubble control unit 125. The gas is subsequently exhausted via an exhaust tube 126 of the antibubble control unit 125 into ambient air or into an exhaust system of the facility. The tubing 124 is preferably made of material that is uncollapsable and airtight. The antibubble control unit comprises a suction device 160 configured to generate a low gas pressure which is propagated via the tube 124 into the gas department 119 of the oxygenator 114. It also comprises a central computer 161 for controlling the performance of the different parts of the system, e.g. the set subatmospheric pressure at a setting device 135 of the gas compartment 119 of the oxygenator 114, the increase in hydrostatic pressure in the blood compartment 117 of the oxygenator 114 or elsewhere, and it may also display parameters of interest on display means 136, 137, and 138 such as pressures, fresh gas oxygen concentration and embolic load.

The oxygenator 114 is further open to ambient air through an opening 127 situated close to the gas outlet 123. This is to prevent a supraatmospheric pressure to develop in the gas compartment 119 in case the gas outlet 123 or gas outlet tubing 124 is unintentionally obstructed. An overpressure in the gas compartment 119 could lead to disastrous air leakage into the blood flow of the arterial line, since the membrane 118 is not always airtight but in most clinical applications composed of a micro-porous material that easily may pass gaseous emboli through the membrane into the liquid, e.g. blood, but does not, due to capillary forces, pass the liquid over the membrane into the gas.

According to embodiments of the present invention, the bubble formation in the blood is to be diminished by lowering the amount of dissolved gases in the blood. This is accomplished by decreasing the gas pressure in the gas compartment 119 of the oxygenator 114. For this purpose a suction apparatus 160 is integrated in the antibubble control unit 125. The suction apparatus 160 can be realized as an ordinary, high-quality suction device that is capable of generating subatmospheric pressures of approximately 0.1 bar. In embodiments of the invention, the suction apparatus 160 is integrated into the antibubble control unit 125, which, in such an embodiment, also contains means to perform the method of the present invention in a safe manner.

The method of the present invention implies that a preset level of vacuum can be maintained in the gas compartment 119 of the oxygenator 114. In order to achieve this, it is desirable that the oxygenator 114 is constructed gas-tight, which means that an anti-overpressurization safety opening 127 must be closed during the vacuum-operation. This safety measure can be accomplished e.g. with a spring type, one-way, valve 128 arranged at the safety opening 127. The valve 128 opens in case of an overpressure and closes in case of a pressure lower than the ambient atmospheric pressure in the gas compartment 119.

The present invention decreases dissolved or comprised gases in the blood. Nitrogen, oxygen, and carbon dioxide and water vapour constitute more than about 99% of these gases under normal circumstances. The organism needs a minimum level of partial pressure of oxygen to sustain aerobic metabolism. Dry air is constituted by approximately 78% nitrogen and 21% oxygen, and the nitrogen is not needed for metabolism. If nitrogen is substituted for oxygen, one can decrease the total gas pressure to 1/5 and still have the same partial pressure of oxygen available for the organism. When vacuum is applied according to the invention, the oxygenator 114 has to be constructed airtight, as mentioned above. Also all connections and tubing 121 of the fresh gas source 120 have to be airtight as depicted in FIG. 1 by the hatched area. The gas outlet tubing 124 is also constructed airtight as needed for any suction apparatus to function.

Today, during conventional extracorporeal circulation in heart surgery, in case there is an air leak in the fresh gas tubing it would perhaps not be noted since the direction of leakage of fresh gas would be out of the oxygenator into the ambient air. However, in case of leakage during vacuum operation there would be an entrance of ambient air (consisting of 78% nitrogen) into the oxygenator, changing the effective level of oxygen in the fresh gas to a lower level. Therefore, the perfusionist has to check for leaks during the procedure and has to monitor the partial pressure of oxygen preferably at the site of gas outlet.

Therefore, in the present invention, an oxygen sensor 129 can be arranged at the gas outlet tubing 124 and configured to monitor the partial pressure of oxygen in the gas leaving the oxygenator 114 via the gas outlet 123. The signal from the oxygen sensor 129 can be diverted to, processed in and presented to a perfusionist by means of the antibubble control unit 125.

According to the invention, the already formed bubbles entering into the oxygenator during the vacuum operation will change in volume proportionally to the decreased total gas pressures exerted on the blood. During the passage through the oxygenator when vacuum is applied, a bubble will thus increase in size. There is formation of a denatured layer of lipoproteins in the gas-blood interface and the transient volume increase of a bubble passing an oxygenator may irreversibly enlarge the total bubble surface of denatured lipoprotein. For example, if bubble volume is increased by 50%, the surface area will increase by approximately 31% (100×(1.5^{1/3})²−100). Even after the disappearance of gas inside a blood bubble the irreversibly denatured surface layer of lipoproteins persists and may form an embolus capable of obstructing capillaries and perhaps also induce a reaction of the body as if it were a foreign body. It may therefore be beneficial to counteract this effect on bubble size due to vacuum application. To this end the present invention may also include the step of increasing transiently the hydrostatic blood pressure in the blood during the passage of the blood compartment of the oxygenator. In clinical practice, though, this may not be deemed necessary.

During clinical perfusion of today, the hydrostatic pressures in the blood tubing before and after the oxygenator 114 is measured by means of pressure sensors 130 and 131, respectively. The pressure measurement is done in order to monitor the pressure gradient generated by the oxygenator 114 and thus to early detect e.g. oxygenator malfunction. In the present invention, the signals from these pressure sensors 130, 131 are fed directly or via a heart-lung machine (not shown) into the antibubble control unit 125. The mean and maximum/minimum pressures in the blood compartment 117 of the oxygenator 114 may be, together with the pressure signal of measured vacuum 139 of the gas compartment 119, used for feedback calculation of appropriate increased hydrostatic pressure that should be applied in the blood compartment 117 of the oxygenator 114 to counteract the chosen level of subatmospheric pressure in the gas compartment 119 of the oxygenator 114 in order to maintain bubble size. Such calculations are performed in a central computer 161 comprised in the antibubble control unit 125.

The blood pressure in the blood compartment 117 of the oxygenator 114 may be measured directly from a location in the compartment 117, or deduced from measurements from other locations 130, 131. The pressure in the blood compartment 117 of the oxygenator 114 can be manipulated or controlled by a clamping device 132 which in turn can be controlled by the antibubble control unit 125. The clamping device 132 is constructed to be able to adjust to very small mechanical changes and to have a small time-constant and hysteresis. The clamping device 132 is preferably easily detachable from the arterial line 115, in case of e.g. malfunction.

A second embodiment of the invention comprises means for the temporary increase of hydrostatic pressure of the blood in the extracorporeal circuit. The purpose of this pressure increasing means is to reduce bubble volume of gas by application of an increased hydrostatic pressure to the bubble-carrying liquid/blood. The increased hydrostatic pressure of blood will be propagated into the gas bubble, thus forcing gas from the bubble into solution, i.e. from gas phase to liquid phase. Subsequently, a new steady state is reached rendering the bubbles smaller not only because of the higher hydrostatic pressure but also from the loss of a portion of the original contained gas of the bubbles that becomes dissolved in the blood. At high enough levels of hydrostatic pressure applied and long enough time period of its application, even a complete annihilation of bubbles may be achieved.

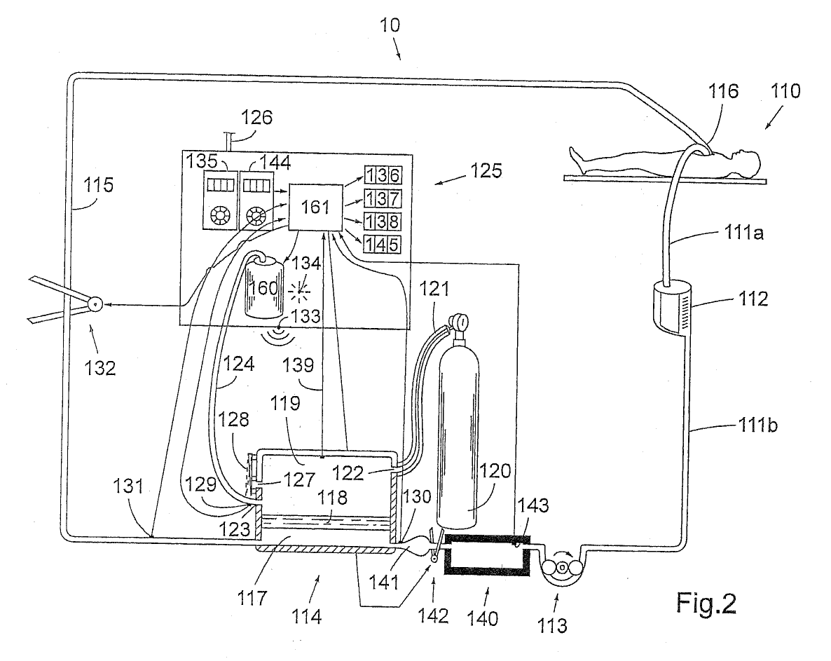

FIG. 2 shows schematically the second embodiment of the invention comprising means for the temporary increase of hydrostatic pressure of the blood in the extracorporeal circuit. In this embodiment, a device for forcing gas contained in bubbles into solution by applying a temporary high hydrostatic pressure to the blood/liquid stream is incorporated. Also, in this embodiment, the pressure increasing means is realized as a high-pressure resistant reservoir 140 for circulating blood. The high-pressure resistant reservoir 140 is preferably arranged between the flow control means 113 and the oxygenator 114. The dimension, e.g. the length, of the tubing between the reservoir 140 and the oxygenator 114 should preferably be minimized in order to allow for a minimum of time period after the pressurization in the high-pressure resistant reservoir 140 before the blood enters the gas-exchanging part 119 of the oxygenator 114. Otherwise, in time, bubbles may regain their former size due to the movement of gas from the supersaturated liquid, just having left the high-pressure resistant reservoir 140, back into existing bubbles, or forming new ones. In this instance it is also important that the outlet 141 from the high-pressure resistant reservoir 140 into ambient atmospheric pressure is hydrodynamically shaped in order to minimize bubble formation due to turbulent flow.

The volume of the high-pressure resistant reservoir 140 is preferably chosen so that enough time is allowed for the redistribution of gas from bubbles into a dissolved state, but also taking into consideration the benefits of minimal priming volumes of the extra-corporeal circuitry. If, for example, a blood flow of 4.5/min and a time period of 10 seconds of high pressurization are needed, then the volume of the high-pressure resistant reservoir 140 should be approximately 0.75 liters. The volume of the reservoir 140 may be reduced when higher pressure is utilized, keeping blood flow constant.

The hydrostatic pressure in the high-pressure resistant reservoir 140 can be manipulated by a clamping device 142 controlled by the antibubble control unit 125 and configured to regulate the outflow resistance from the high-pressure resistant reservoir 140. The clamping device 142 is further constructed to be able to adjust to very small mechanical changes and to have a small time-constant and hysteresis. Also, it has to be easily detachable from the arterial line, in case of malfunction. In this embodiment of the invention, a pressure sensor 143 is arranged at the high-pressure resistant reservoir 140. The pressure sensor 143 is configured to register the pressure in the reservoir 140 and transmit a registered pressure value as a pressure signal to the antibubble control unit 125. In the central computer 161 of the antibubble control unit 125, the pressure value is compared to a set value 144 chosen by the perfusionist. Further, the central computer 161 generates an appropriate control signal by means of which signal the operation of the clamping device 142 is controlled, whereby a desired pressure level can be achieved in the high-pressure resistant reservoir 140. The signal from the pressure monitor 143 is preferably presented to the perfusionist on the antibubble control unit 125 display.

The antibubble control unit 125 in the present invention contains the function to generate subatmospheric pressures by a suction device 160 contained in the antibubble control unit 125 and to generate increased hydrostatic pressure in the blood compartment 117 of the oxygenator 114. The central computer 161 of the antibubble control unit 125 can also be configured to calculate transmembrane pressure over the oxygenator membrane 118, and to alert an alarm signal by means of a sound alarm 133 and/or by means of a visible alarm 134 when reaching non-allowable limits.

The antibubble control unit 125 can be configured to comprise a setting device 135 by means of which a perfusionist is able to enter a chosen level of subatmospheric pressure desired in the gas compartment 119. The antibubble control unit 125 can further comprise a computer 161 configured to calculate, based on the preset level of vacuum, an appropriate level of increased pressure in the blood compartment 117 in order to avoid enlargement of preformed bubbles entering the oxygenator 114. The pressure signals registered by the pressure sensors 130, 131 over the blood compartment 117 can be used for electronic feedback control of the adjustment of the clamp 132 to generate an appropriate increased level of pressure in the blood compartment 117. The antibubble control unit 125 can also contain one or more displays 136, 137, and 138 for presenting for example actual measured levels of gas compartment pressure 139, blood compartment pressure 130, 131 and gas flow oxygen concentration 129, respectively. These presented parameters or, when appropriate, their calculated differences can all be connected into one or several common or separate alarm devices 133, 134.

In the second embodiment of the invention, the antibubble control unit 125 further comprises means configured to feed-back regulate the pressure in a high-pressure resistant reservoir 140. The feed-back means can comprise setting means 144 for setting the pressure level in the high-pressure resistant reservoir 140 and display means 145 to monitor the generation of the pressure in the high-pressure resistant reservoir 140.

It is important to be able to measure changes in bubble formation when the methods and equipment according to this invention are employed. In a third embodiment of the invention, the inventive system contains means to monitor and document the occurrence of bubbles more properly than currently employed. Heart-lung machines contain bubble monitoring devices with sensors to be attached to the tubing and which are configured to warn the perfusionist when bubbles appear and they may also be configured to automa-tically halt the main pump in case larger bubbles occur. The sensitivity of the bubble sensors of heart-lung machines in common use, e.g. in Jostra HLM 20 and Stockert S3, is 300 micrometers, which is to be compared with the size of blood capillaries which may be in the range of the diameter of a single red blood cell i.e. 7 micrometers. The bubble detecting device already equipped into a heart-lung machine may therefore be too insensitive.

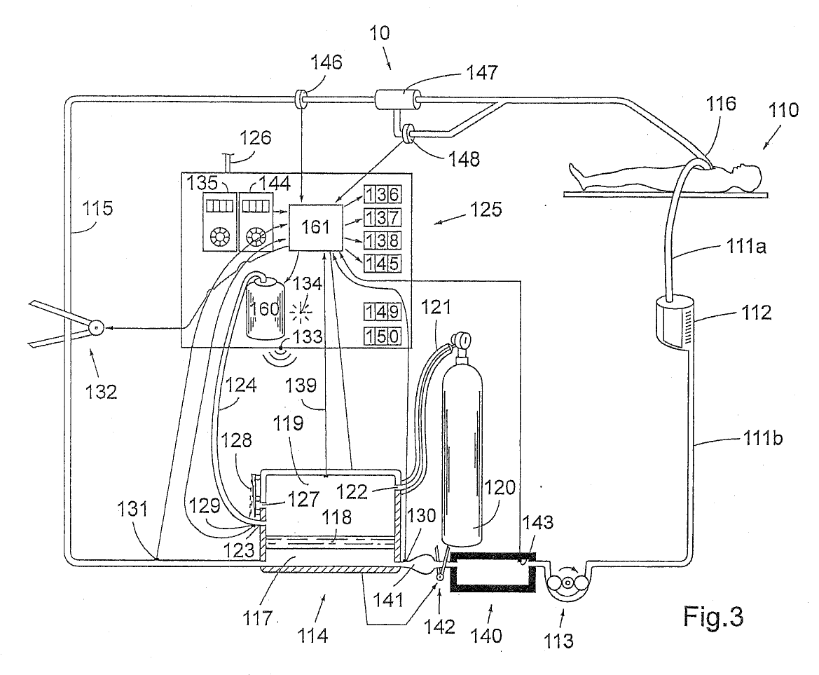

FIG. 3 shows schematically a third embodiment of the present invention. In this embodiment one or several bubble sensors for quality control are attached directly to the arterial line 115 and/or to a tube containing filtered plasma from the blood of the arterial line. As illustrated in FIG. 3, a high sensitivity first bubble sensor 146 is attached to the arterial line 115 and communicatively connected to the antibubble control unit 125. The high sensitivity bubble sensor 146 could for example be realized as a bubble detector with sensitivity down to sizes when the corpuscular elements of the blood come into play i.e. about 10-15 micrometers. The signal from the bubble sensor is handled by the central computer 161 of the antibubble control unit 125 which may show the occurrence of gas emboli in a display means, sound or light an alarm, or even halt the pump.

Sensors of higher sensitivity than mentioned above may be functional only with filtered blood i.e. blood plasma, where no formed elements of blood such as red or white blood cells or platelets appear. Thus in order to be able to use such sensors, it may be necessary to incorporate a blood filtering device 147 from which blood plasma is bypassed and sensed for emboli by a second bubble sensor 148 having a higher accuracy, discerning bubbles down to fractions of a micrometer.

The size and frequency of occurrence of the bubbles may be presented to the perfusionist visually on display means 137, 138 and/or audibly by means of audible means 133 and on a display of the antibubble control unit 125, forcing the perfusionist to take appropriate action.

The present invention also relates to a kit containing disposable articles comprises one or several pressure measurement tubes according to the specifications above, configured to be attached to the measurement outlets of the blood tubing and oxygenator, respectively. The kit can further comprise a gas-tight oxygenator and optionally a high-pressure resistant reservoir.

The present invention has been described in detail above but it is obvious to a person skilled in the art that the invention may be modified in other ways within the scope of the appended claims.

1. A system for controlling the amount and size of bubbles and/or gas comprised in a fluid flowing in an extracorporeal circuit, comprising:
a fluid supply means (111a) configured to supply a fluid to the extracorporeal circuit (111a, 111b, 113, 114, 115, 116);
a flow control means (113) connectable to the extracorporeal circuit and configured to control the flow of the fluid in the extracorporeal circuit;
a gas exchange means (114) connectable to the extracorporeal circuit and configured to diminish and/or exchange gas comprised in the fluid circulated in the extracorporeal circuit;
an antibubble control unit (125) connectable to an outlet (123) of the gas exchange means (114) and configured to control the total gas pressure over a gas-fluid separating membrane (118) of the gas exchange means (114), whereby the comprised amount of gas in the fluid leaving the gas exchange means (114) can be controlled; and
fluid return means (115, 116) connectable to the gas exchange means (114) and configured to reintroduce the fluid into the patient.

2. The system of claim 1, further comprising a pressure increasing means (140) connected to the extracorporeal circuit preferably between the flow control means (113) and the gas exchange means (114), the pressure increasing means (140) being configured to apply a high hydrostatic pressure to the fluid during its passage through said pressure increasing means (140), whereby gas contained in bubbles is forced into a dissolved state.

3. The system of claim 2, wherein the volume of the pressure increasing means (140) is chosen to allow required time for the redistribution of gas from bubbles into a dissolved state, and wherein the pre-selected pressure of the pressure increasing means (140) is dependent on the volume in said pressure increasing means (140) and the flow rate of the fluid flowing in the extracorporeal circuit, and wherein the pressure increasing means (140) comprises an outlet (141) connecting the pressure increasing means (140) to an inlet of the oxygenator (114), which outlet (141) is hydrodynamically shaped in order to minimize bubble formation during pressure normalization.

4. The system of claim 3, further comprising a first clamping device (142) arranged at the outlet (141) of the pressure increasing means (140), controlled by the antibubble control unit (125) and configured to regulate the pressure in the pressure increasing means (140) by regulating the flow resistance out from the pressure increasing means (140).

5. The system of claim 4, further comprising a pressure sensor (143) arranged at the pressure increasing means (140), and configured to register the pressure in the pressure increasing means (140) and to transmit a registered pressure value as a pressure signal to the antibubble control unit (125); which antibubble control unit (125) is configured to compare the pressure value with a preset value and to generate a control signal by means of which control signal the operation of the first clamping device (142) can be controlled, whereby a desired pressure level can be achieved in the pressure increasing means (140).

6. The system of claim 1, wherein the gas exchange means (114) is realized as an air-tight oxygenator (114) comprising a first compartment (117) connected to a blood inlet and outlet of the oxygenator (114), a gas-fluid separating membrane (118) through which the gas exchange occurs, and a second compartment (119) comprising a gas inlet and outlet of the oxygenator (114).

7. The system of claim 6, wherein the antibubble control unit (125) is configured to generate a subatmospheric pressure in the gas compartment (119) of the oxygenator (114) by means of a suction device (160) which may be incorporated in the antibubble control unit (125) and/or to generate an increased hydrostatic pressure in a blood compartment (117) of the oxygenator (114), whereby the enlargement of bubbles entering the blood compartment (117) of the oxygenator (114) during vacuum operation is counteracted.

8. The system of claim 6, further comprising a gas source (120) configured to supply fresh gas to the second compartment (119) of the oxygenator (114) via a gas supply tube (121) and a gas inlet (122) of the oxygenator (114), and wherein the separating membrane (118) is permeable to the supplied components of fresh gas, and wherein the oxygenator (114) is configured such as the partial pressures of gas in the second compartment (119) and the partial pressures of the gas dissolved in the fluid in the first compartment (117) tend to equalize, whereby gas exchange between the fresh gas and the fluid occurs.

9. The system of claim 6, wherein the gas outlet (123) of the oxygenator (114) is connected to the antibubble control unit (125) by means of an uncollapsable tubing (124), whereby gas flows from the second compartment (119) of the oxygenator (114) to the antibubble control unit (125) and is subsequently exhausted from the antibubble control unit (125) via an exhaust tube (126).

10. The system of claim 9, wherein a safety opening (127) to ambient air is arranged at the oxygenator (114) close to the gas outlet (123), the safety opening (127) being configured to prevent an overpressure in the second compartment (119) in case the gas outlet (123) or outlet tubing (124) is obstructed, and wherein the oxygenator (114) is constructed air-tight, the safety opening (127) is closeable by means of a valve (128), and wherein a preset level of vacuum is maintained in the second compartment (119), whereby the lowered amount of comprised gas in the fluid diminishes bubble formation in the fluid during the passage through the extracorporeal circuit and possibly in the body.

11. The system of claim 1, further comprising pressure sensors (130, 131, 139) arranged to measure the pressures in the fluid tubings before and after the gas exchange means (114), respectively, or directly in the blood compartment (117) and in the gas compartment (119); and to transmit the pressure measurements to the antibubble control unit (125), which unit (125) is configured to monitor the pressure gradient generated over the gas-fluid separating membrane (118) and configured to indicate when transmembrane pressure gradients approach non-allowable limits.

12. The system of claim 11, further comprising a second clamping means (132) arranged at the fluid return means (115), said second clamping means (132) being controlled by the antibubble control unit (125) and configured to regulate the hydrostatic pressure in the first compartment (117) of the gas exchange means (114) whereby bubbles contained in the blood passing the blood compartment of the oxygenator (117) during vacuum operation keep their size by feedback control based on the blood pressure measurement from the blood compartment (117) and the gas pressure measurement (139) of the gas compartment (119) of the oxygenator (114).

13. The system of claim 1, further comprising a first bubble sensor (146) arranged at the fluid return means (115) and connected to the antibubble control unit (125), the first bubble sensor (146) is configured to detect an embolus or a bubble in said fluid return means (115) and, by control imposed by the computer included in the antibubble control unit (125), according to the size and frequency of bubbles occurring, appropriately display bubble presence, light and/or sound an alarm signal or halt the main pump of the heart-lung machine or another kind of extracorporeal fluid device.

14. The system of claim 13, further comprising a filtering device (147) arranged at the fluid return means (115), by means of which filtering device (147) a fluid part can be bypassed and sensed for bubbles or emboli by a second bubble sensor (148) and display bubble presence, light and/or sound an alarm signal or halt the main pump of the heart-lung machine or another kind of extracorporeal fluid device.

15. The system of any of claim 1, wherein the antibubble control unit (125) comprises display means (136, 137, 138, 145, 149, 150) configured to present operation parameters or sensor measurements, alarm devices (133, 134) configured to alert a sound alarm and/or a visible alarm when e.g. non-acceptable limits are reached; and comprises further interactive means (135, 144) configured to let a user enter desired operational parameters.

16. A method for controlling the amount and size of bubbles and/or gas comprised in a fluid flowing in an extracorporeal circuit, comprising the steps of:
providing a fluid supply means (111a) configured to supply a fluid to the extracorporeal circuit (111a, 111b, 113, 114, 115, 116);
connecting a flow control means (113) to the extracorporeal circuit, the flow control means (113) being configured to control the flow of the fluid in the extracorporeal circuit;
connecting a gas exchange means (114) to the extracorporeal circuit, the gas exchange means (114) being configured to diminish and/or exchange gas comprised in the fluid circulated in the extracorporeal circuit;
connecting an antibubble control unit (125) to an outlet (123) of the gas exchange means (114), the antibubble control unit (125) being configured to control the total gas pressure over a gas-fluid separating membrane (118) of the gas exchange means (114), whereby the comprised amount of gas in the fluid leaving the gas exchange means (114) can be controlled; and
connecting a fluid return means (115, 116) to the gas exchange means (114), the fluid return means (115, 116) being configured to reintroduce the fluid into the patient.

17. The method of claim 16, further comprising the step of providing a pressure increasing means (140) connected to the extracorporeal circuit preferably between the pumping means (113) and the oxygenator (114), the pressure increasing means (140) being configured to apply a high hydrostatic pressure to the fluid during its passage through said pressure increasing means (140), whereby gas contained in bubbles is forced into a dissolved state.

18. The method of claim 17, further comprising the steps of selecting the volume of the pressure increasing means (140) to allow required time for the redistribution of gas from bubbles into a dissolved state, wherein the pre-selected pressure of the pressure increasing means (140) is dependent on the volume in said pressure increasing means (140) and the flow rate of the fluid flowing in the extracorporeal circuit, and by means of an outlet (141) connecting the pressure increasing means (140) to an inlet of the oxygenator (114), which outlet (141) is hydrodynamically shaped in order to minimize bubble formation during pressure normalization.

19. The method of claim 18, further comprising the step of providing a first clamping device (142) at the outlet (141) of the pressure increasing means (140), the clamping device (142) being controlled by the antibubble control unit (125) and configured to regulate the pressure in the pressure increasing means (140) by regulating the flow resistance out from the pressure increasing means (140).

20. The method of claim 19, further comprising the step of providing a pressure sensor (143) at the pressure increasing means (140), which pressure sensor (143) registers the pressure in the pressure increasing means (140) and transmits a registered pressure value as a pressure signal to the antibubble control unit (125); which antibubble control unit (125) compares the pressure value with a preset value and generates an appropriate control signal by means of which the operation of the clamping device (142) is controlled, whereby a desired pressure level can be achieved in the pressure increasing means (140).

21. The method of claim 16, wherein the gas exchange means (114) is realized as an air-tight oxygenator (114) comprising a first compartment (117) connected to a blood inlet and outlet of the oxygenator (114), a gas-fluid separating membrane (118) through which the gas exchange occurs, and a second compartment (119) comprising a gas inlet and outlet of the oxygenator (114).

22. The method of claim 21, further comprising the steps of generating a subatmospheric pressure in the gas compartment (119) of the oxygenator (114) by means of a suction device which may be incorporated in the antibubble control unit (125) and/or to generate an increased hydrostatic pressure in a blood compartment (117) of the oxygenator (114), whereby the enlargement of bubbles entering the blood compartment (117) of the oxygenator (114) during vacuum operation is counteracted.

23. The method of claim 21, further comprising the steps of supplying fresh gas to the second compartment (119) of the oxygenator (114) via a gas supply tube (121) and a gas inlet (122) of the oxygenator (114), and equalizing the partial pressures of gas in the second compartment (119) and the partial pressures of the gas dissolved in the fluid in the first compartment (117), whereby gas exchange between the fresh gas and the fluid occurs.

24. The method of any of claim 21, further comprising the step of connecting the gas outlet (123) of the oxygenator (114) to the antibubble control unit (125) by means of an uncollapsable tubing (124), whereby gas flows from the second compartment (119) of the oxygenator (114) to the antibubble control unit (125) and is subsequently exhausted from the antibubble control unit (125) via an exhaust tube (126).

25. The method of claim 24, further comprising the step of providing a safety opening (127) to ambient air at the oxygenator (114) close to the gas outlet (123), the safety opening (127) being configured to prevent an overpressure in the second compartment (119) in case the gas outlet (123) or outlet tubing (124) is obstructed, and maintaining a preset level of vacuum in the second compartment (119), whereby the lowered amount of dissolved gas in the fluid diminishes bubble formation in the fluid during the passage through the extracorporeal circuit and possibly in the body.

26. The method of claim 16, further comprising the step of providing pressure sensors (130, 131, 139) to measure the pressures in the fluid tubings before and after the oxygenator (114), respectively, or directly in the blood compartment (117) and in the gas compartment (119); and to transmit the pressure measurements to the antibubble control unit (125), which unit (125) monitors the pressure gradient generated over the gas-fluid separating membrane (118) and configured to indicate when transmembrane pressure gradients approach non-allowable limits.

27. The method of claim 26, further comprising a second clamping means (132) arranged at the fluid return means (115), said second clamping means (132) being controlled by the antibubble control unit (125) and configured to regulate the hydrostatic pressure in the first compartment (117) of the oxygenator (114) whereby bubbles contained in the blood passing the blood compartment of the oxygenator (117) during vacuum operation keep their size by feedback control based on the blood pressure measurement (130, 131) of the blood compartment (117) and the gas pressure measurement (139) of the gas compartment (119) of the oxygenator (114).

28. The method of any of claim 16, further comprising the step of providing a first bubble sensor (146) at the fluid return means (115), the first bubble sensor (146) being connected to the antibubble control unit (125) and configured to detect an embolus or a bubble in said fluid return means (115) and, by control imposed by the computer included in the antibubble control unit (125), according to the size and frequency of bubbles occurring, appropriately display bubble presence, light and/or sound an alarm signal or halt the main pump of the heart-lung machine or another kind of extracorporeal fluid device.

29. The method of claim 28, further comprising the step of providing a filtering device (147) at the fluid return means (115) from which filtering device (147) a fluid part can be bypassed and sensed for bubbles or emboli by a second bubble sensor (148) and display bubble presence, light and/or sound an alarm signal or halt the main pump of the heart-lung machine or another kind of extracorporeal fluid device.

30. The method of claim 16, further comprising the steps of presenting operation parameters or sensor measurements on display means (136, 137, 138, 149, 150), of alerting a sound alarm and/or a visible alarm, when e.g. non-allowed limits are reached, on alarm devices (133, 134); of letting a user enter desired operation parameters on an interactive means (135, 144).

31. An anti bubble control unit (125) for use in a system according to claim 1.

32. A gas exchange means (114) air-tight and comprising a safety valve (127) for use in a system according to claim 1.

33. A pressure increasing means (140) for use in a system according to claim 1, said high pressure resistant reservoir being configured to force existing gas in bubbles into solution during the extracorporeal circulation procedure.
